(19) United States
(12) Patent Application Publication
(10) Pub. No.: US 2008/0234942 A1
(43) Pub. Date: Sep. 25, 2008

(54) Analyzing Body Tissue

(75) Inventors: Matthew Gaved, Cambridge (GB); Michael Farquharson, London (GB)

Correspondence Address: ARENT FOX LLP, 1050 CONNECTICUT AVENUE, N.W., SUITE 400, WASHINGTON, DC, 20036, US

(73) Assignee: TISSUOMICS LIMITED, Cambridge (GB)

(21) Appl. No.: 11/630,251

(22) PCT Filed: Jun. 27, 2005
(86) PCT No.: PCT/GB2005/002511

§ 371 Date: Jun. 11, 2008

(30) Foreign Application Priority Data
Jun. 25, 2004 (GB) 0414318.6

(51) Int. Cl. G01N 1/30; C12M 1/34; G06F 19/00; G06F 17/10

(52) U.S. Cl. 702/19; 435/40.52; 435/287.1; 703/2

(57) ABSTRACT

The invention describes a method for analysing a body tissue sample, the method comprising irradiating the tissue sample (4) with penetrating radiation and detecting transmitted, (14) and/or scattered radiation (16, 18) from the sample, wherein the duration of the analysis is determined from one or more desired confidence levels based on a model or algorithm defining a relationship between analysis duration and confidence level. The invention also describes apparatus for implementing the method and computer software for controlling the apparatus.

### FIELD OF THE INVENTION

The present invention relates to methods and systems for analysing body tissue. The invention has particular, although not necessarily exclusive, application in the characterisation of body tissue, for instance characterisation of tissue as normal (e.g. healthy) or abnormal (e.g. pathological). It is useful in the diagnosis and management of cancer, including breast cancer.

### BACKGROUND

In order to manage suspected or overt breast cancer, tissue is removed from the patient in the form of a biopsy specimen and subjected to expert analysis by a histopathologist. This information leads to the disease management program for that patient. The analysis requires careful preparation of tissue samples that are then analysed by microscopy for prognostic parameters such as tumour size, type and grade. An important parameter in tissue classification is quantifying the constituent components present in the sample. Interpretation of the histology requires expertise that can only be learnt over many years based on a qualitative analysis of the tissue sample, which is a process prone to intra and inter observer variability.

Despite the relative value of histopathological analysis, there remains a degree of imprecision in predicting tumour behaviour in the individual case. Additional techniques have the potential to fine-tune tissue characterisation to a greater degree than that currently used and hence will improve the targeted management of patients.

In existing research in this field, x-ray fluorescence (XRF) techniques have been used to study trace element composition of breast tissue and have shown that breast cancer is accompanied by changes in trace elements and such measurements could contribute to tissue grading. It has also been shown that x-ray diffraction effects can operate as an effective means of distinguishing certain types of tissue. Furthermore, it has been shown that such diffraction effects could be suitably analysed to demonstrate small differences in tissue components and that this analysis could lead to a quantitative characterisation of tissues.

In co-pending PCT patent application PCT/GB04/005185 we describe an approach to characterising body tissue samples, in which tissue characteristics are modelled using a multivariate model. The inputs to the model can include a variety of measured tissue properties and measurements derived using x-rays and/or other penetrating radiations, including for example, x-ray fluorescence (XRF), Compton scatter and/or Compton scatter densitometry, energy dispersive x-ray diffraction (EDXRD), angular dispersive x-ray diffraction (including wide angle x-ray scattering (WAXS), low angle x-ray scattering, small angle scattering (SAXS), and ultra low angle scattering (ULAX) and linear attenuation (transmission). In co-pending PCT patent applications PCT/GB05/001987 and PCT/GB05/002002 we describe apparatus that can be used to capture these measurements. In co-pending PCT patent application PCT/GB05/001999 we describe an ‘intelligent’ scanning system that can be employed to optimise the payoff between the information content of the measurements and dose, which is of particular relevance to in vivo measurements. One factor that affects dose is the duration of time for which any particular tissue region is exposed to the e.g. X-ray radiation. By minimising the duration of any exposure period, the dose can likewise be limited.

When taking in vitro measurements, there are not the same dose limitation considerations that exist for in vivo measurements. There is still, however, a desire to minimise the time taken to analyse a tissue sample, e.g. to minimise any delay in returning results to a clinician and/or to increase the rate at which samples can be processed by a facility.

### SUMMARY OF THE INVENTION

The present invention is concerned, in general terms, with approaches to minimising the duration of a tissue analysis (e.g. characterisation) process, whilst maintaining a desired level of confidence in the results that are obtained.

The approach adopted by the invention is to determine for the analysis of each particular tissue sample an optimal duration for the analysis to achieve a desired confidence level, the optimal duration being determined based on empirically derived models or algorithms defining a relationship between analysis duration and confidence level.

Accordingly, in one aspect the invention provides a method for analysing a body tissue sample, the method comprising irradiating the tissue sample with penetrating radiation (e.g. X-ray radiation) and detecting transmitted and/or scattered radiation from the sample, wherein the duration of the analysis is determined from one or more desired confidence levels based on a model or algorithm defining a relationship between analysis duration and confidence level.

The desired confidence level or levels may be operator selected or automatically set.

The determination of duration may be a one-off calculation at the start of the analysis procedure (or at some predetermined point in the procedure). More preferably, however, the duration is adjusted dynamically as the analysis progresses, or is at least updated one or more times during the course of the analysis.

The model(s) and/or algorithm(s) preferably take into account (and as a result the duration calculation is based on) a number of factors including, for example:
histopathology data and/or histopathological diagnosis of tissue samples, such whether the tissue exhibits benign or malignant change etc;
tissue characteristics/types (e.g. adipose, glandular or fibrous; normal, abnormal benign, abnormal malignant);
patient information (e.g. medical history, family history, age etc), but also other patient information such as previous or current treatments the patient has undergone. For instance, if the patient had pre-operative chemotherapy to reduce the size or proliferation of the tumour, it is likely that this would have an effect on the cellular composition and, thus, almost certainly the x-ray scattering signature. For this reason it may be advantageous or preferable to take into account recent and/or prior treatment information in the analysis and processing of data obtained from patient tissue samples and in the training model(s) and/or algorithms; and
Other tissue information more generally. Such “other tissue” information may include information about the genomic or proteomic composition/profile of the tissue. Although, genomic or proteomic data would not necessarily be used as an immediate or relatively immediate parameter for the model(s) and/or algorithm(s), it may be utilised to train the model(s) and/or algorithm(s) and/or be used in their development and/or used on a sample for in vitro analysis.

Some of these factors may themselves become better defined or known for the first time as the analysis progresses (e.g. characterisation of the tissue as normal/abnormal) and the calculated duration can be modified accordingly.

In other aspects, the invention provides methods for creating and/or updating models and/or algorithms defining a relationship between analysis duration and confidence level. The models or algorithms are preferably derived empirically, based on a large number of measurements from tissue samples exhibiting a variety of factors.

The invention also provides tissue analysis apparatus and systems that can be operated in accordance with the methods discussed above, and software for controlling such apparatus and systems in this manner.

### BRIEF DESCRIPTION OF THE DRAWINGS

Embodiments of the invention are described below by way of example with reference to the accompanying drawings, in which:

FIG. 1 is a schematic illustration of in vitro X-ray tissue analysis apparatus operable in accordance with embodiments of the present invention;

FIG. 2 illustrates a tissue analysis process in accordance with a first embodiment of the present invention;

FIG. 3 illustrates a tissue analysis process in accordance with a second embodiment of the present invention;

FIG. 4 illustrates a tissue analysis process in accordance with a third embodiment of the present invention;

FIG. 5 illustrates possible theoretical relationships between analysis duration and diagnostic accuracy; and

FIG. 6 illustrates the payoff between analysis duration and diagnostic accuracy for four different tissue types.

### DESCRIPTION OF EMBODIMENTS

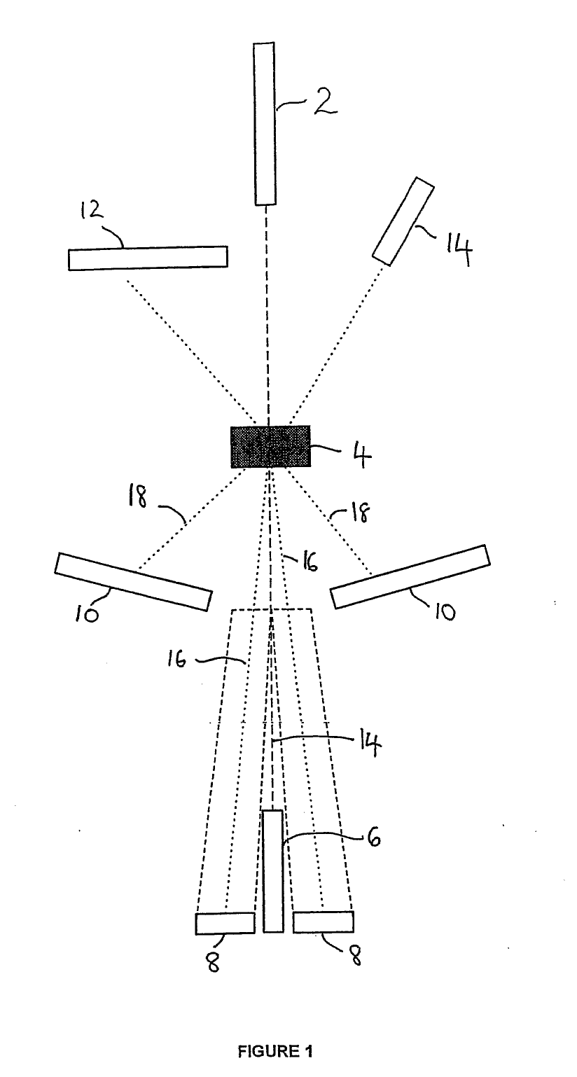

FIG. 1 illustrates an apparatus suitable for in vitro irradiation of a tissue sample (e.g. a breast tissue sample that has been obtained from a biopsy). The apparatus comprises a penetrating radiation (in this example X-ray) beam source 2 that directs a beam of X-ray radiation onto the tissue sample 4 being examined. A series of detectors 6, 8, 10, 12, 14 are arranged below and above the sample 4 to detect both transmitted and scattered X-ray radiation.

Looking in more detail at the detector arrangement illustrated in FIG. 1, it can be seen that below the sample 4 there are two of pairs of detectors 8,10 arranged to detect scattered radiation 16,18 and a single detector 6 for detecting transmitted radiation 14. The detectors 8 are for detecting ultra-low angle scatter (around 1 degree). The detectors 10 are for detecting wider angle scatter (of about 5 to 8 degrees in the present example).

Above the sample, there is a detector 12 for detecting Compton scatter at high angles (about 120 degrees and more) and an XRF detector 14.

In use, the tissue sample 4 is irradiated by the X-ray source 2 and measurements collected by one or more of the detectors are recorded and processed to obtain a characterisation of the tissue sample to a desired confidence level. The characterisation of the tissue may, for example, be to distinguish normal from abnormal tissue, fibrous from adipose, malignant from benign, any combination of these, or other tissue characteristics.

The characterisation of the tissue can be accomplished, for example, by using a multivariate model such as the one described in PCT patent application PCT/GB04/005185.

The remainder of this description focuses on the approaches that can be taken, in accordance with embodiments of the invention, to controlling the analysis process to obtain a desired confidence level (i.e. accuracy in terms of sensitivity and/or specificity) whilst minimising the duration of the analysis.

One approach that can be used in an attempt to ensure a desired confidence level is obtained is simply for the duration of the analysis to be chosen and fixed for all tissue samples at a time period that, from observing past tests, is more than sufficient to achieve the desired confidence level irrespective of the nature of the sample. In practice, however, this will mean that the duration of the analysis is excessive (i.e. longer than is necessary to achieve a desired confidence level) in many cases. For in vitro tests, this has an impact, for example, on the speed with which results can be provided to a clinician and the rate at which samples can be analysed by any particular testing facility. For in vivo tests, excessive duration has the added disadvantage that the resultant dose delivered to the patient is higher than it need be.

For example, by way of illustration only, say the duration of acquisition of data from an in vitro analysis in a particular test is 60 minutes. The question is whether a longer duration would materially increase the accuracy (confidence level) of the analysis, or conversely whether a shorter duration would materially decrease the accuracy. Say for example that the confidence level (accuracy) at 60 minutes is 95%, and that after a further 60 minutes it has only increased to 96%, there is little value in the extended duration analysis; there is a very poor payoff between additional time and quality of information.

In practice, particularly if using two or more types of measurement (as described for example in co-pending PCT patent application PCT/GB04/005185), useful information may be obtained much earlier than 60 minutes.

Again, purely as an illustration, it might be that:
(1) Within 20 minutes the data is sufficient to diagnose to a high degree of confidence that the tissue was not adipose;
(2) After another 10 minutes (total 30) to confirm (at a higher level of confidence) that the tissue was glandular/fibrous;
(3) After a further 15 minutes (total 45), there is a 75% confidence level that the tissue is abnormal; and
(4) After the full 60 minutes, there is a 95% confidence that the tissue is abnormal but not malignant; but
(5) After a further 60 minutes (as above), the confidence/accuracy only increases to 96%.

In practice, the relationship between tissue typing/characterisation and diagnosis confidence levels and time of exposure to the X-ray source will depend on many factors—which typically will have to be determined through empirical research.

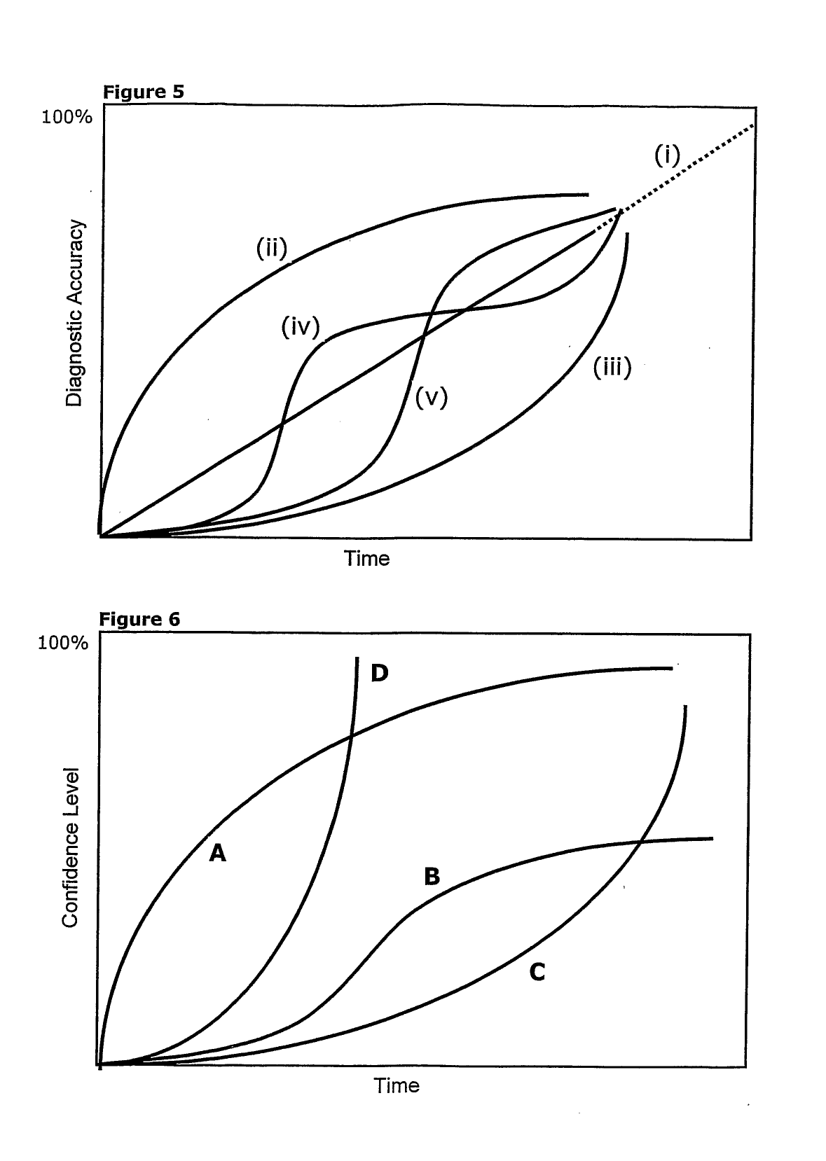

FIG. 5 illustrates, once again by way of illustration only, five possible theoretical relationships (models) between analysis duration (horizontal axis) and diagnostic accuracy/confidence level (vertical axis). In each case, the quality of information increases over time and trends towards 100% (ie 100% is absolute confidence that the diagnosis is correct).

In patterns (i) to (iii), the relationship between diagnostic value and time is continuous and smooth; there are no discontinuities. In pattern (i) there is a linear relationship so that each minute of exposure increases the accuracy of the result by the same amount. This is extremely unlikely in practice.

Patterns (ii) and (iii) and (iv) shown non-linear smooth curve relationships.

In model/curve (ii), most of the diagnostic information is available early on in the exposure cycle. In this model, reducing the exposure time by 50% from 60 mins to 30 mins may not significantly reduce the confidence level for some diagnostic requirements (e.g. discriminating between adipose and glandular/fibrous—as in (2) above for example).

Pattern (iii) shows that most of the diagnostic value is achieved in the late stages of the exposure cycle; there is very little discrimination between tissue types in the first 30-45 minutes, and most discrimination is achieved in the last 15 minutes of the 60 minute cycle.

Pattern (iv) is a combination of (ii) and (iii). Most of the diagnostic value is obtained at around 45 minutes and the sharpest payoff between time and diagnostic value is obtained between 30 and 45 minutes. The minimum acceptable exposure time is likely to be 30 minutes, but there is not much value in exposing for more than 45 minutes.

Pattern (v) is more complex, but likely to be closer to the real world—some discriminations will be achieved significantly ahead of others, at least in part because of the different levels of confidence that may need to be achieved or are desired. This is the model set out in (1) to (5) above. The required confidence for (say) adipose is achieved after 20 minutes, but exposure of 45 minutes is required to differentiate (and therefore differentially diagnose) between abnormal benign and malignant glandular tissue.

Complex patterns like (v) in FIG. 5 and others in practice in the real world are therefore likely to be composed of two or more diagnostic payoffs between different tissue types. This principle is illustrated in FIG. 6 for four tissue types/characteristics, A, B, C and D. Embodiments of the present invention, three examples of which are described below, are generally concerned with optimising the duration of any particular analysis (and in some cases also selecting the detectors used for the measurements taken during the analysis) based on factors including, for instance, the diagnosis required (i.e. which tissue characteristics it is hoped to distinguish), desired confidence levels (which may be different for different tissue types/characteristics) and the tissue type. In the preferred embodiments these factors are taken into account dynamically as the analysis progresses. For example, as the analysis progresses, more may be learnt about the tissue type and the duration and/or desired confidence levels manipulated accordingly.

Models such as those discussed above can be created and modified over time, in accordance with aspects of the present invention, based on empirical measurements and observations from a variety of tissue samples. These models can then be used, in accordance with other aspects of the invention, to determine for any particular tissue analysis the duration required to achieve the desired result. This duration may be determined up front when the analysis is initiated or may be dynamically determined as the analysis progresses.

Similarly, based on these or other empirical models or data, the particular measurements taken at various stages through an analysis cycle can be predetermined or controlled dynamically.

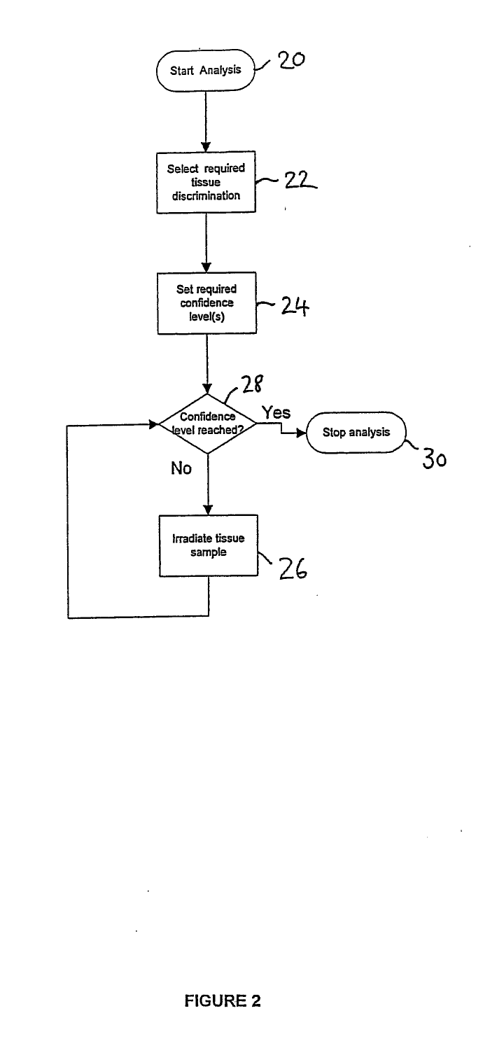

We turn now to the exemplary processes illustrated in FIGS. 2, 3 and 4, starting with FIG. 2.

In the process illustrated in FIG. 2, when the analysis is initiated 20, the operator first selects the tissue discrimination, i.e. diagnosis, type that is to be determined (or this may be pre-defined) 22. For example, the aim may be to characterise a tissue sample as normal, abnormal benign or abnormal malignant. Alternatively or additionally, it may be desired to determine whether the tissue is predominantly adipose or fibrous or to determine the relative ratios of these tissue types in the sample. Other diagnoses (e.g. tissue characterisations) are possible.

The operator also selects the desired confidence level (i.e. accuracy) for the chosen diagnosis 24. Alternatively, the confidence level may be pre-determined and set automatically based, for example, on the chosen diagnosis. In this latter case, the operator is preferably able to override the pre-set value to select a different confidence level if they choose.

The irradiation of the sample 26 then commences and continues until it is determined that the desired confidence level in the chosen diagnosis is obtained 28, at which point the analysis stops 30.

This approach can be extended to base the completion of the diagnosis on multiple confidence levels associated with multiple differential identification of tissue types, which leads (more or less directly) to a differential diagnosis.

For instance in a “two tissue” model, where the payoffs are distinct by time and confidence level, this approach can be implemented quite simply. For example:
1) The operator sets (a) a first discrimination required and (b) confidence level for first tissue discrimination—eg setting (a) adipose or glandular/fibrous and (b) 95% confidence.
2) The operator repeats this selection for a second discrimination with a second confidence level - e.g. setting (c) benign or malignant (d) 75% confidence.

It will be readily apparent how this approach can be adapted to the manual setting for ‘n’ tissue types and a corresponding ‘m’ confidence levels, where ‘n’ and ‘m’ are any integer number (generally, but not necessarily, the same number).

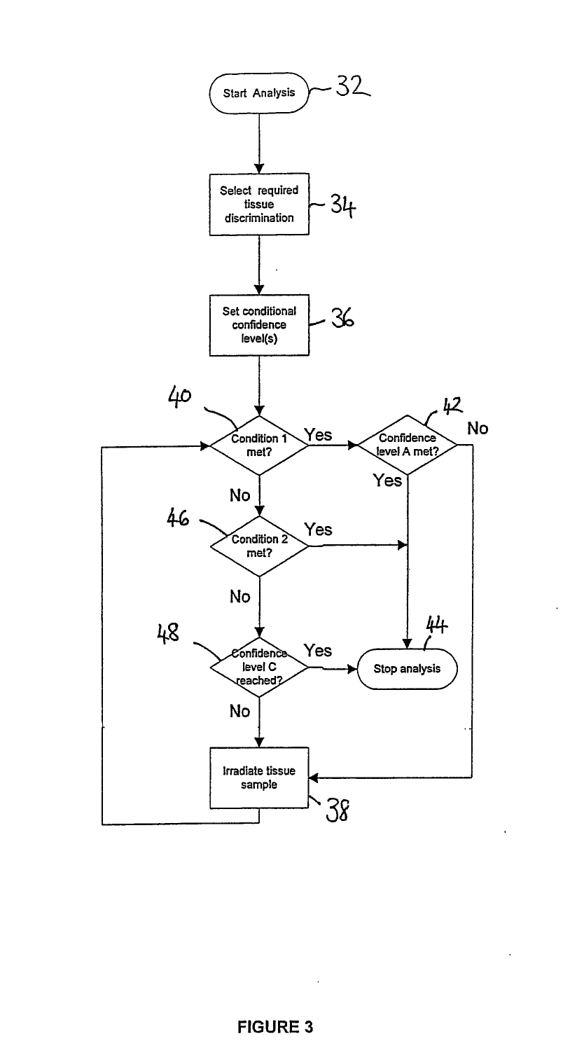

FIG. 3 illustrates another analysis process in accordance with an embodiment of the invention. In this process, the confidence level to be attained is conditional on one or more other factors.

By way of example, the analysis could involve conditional requirements such that:
IF the tissue is identified as Adipose, provide result after 95% confidence level reached and stop procedure (allowing machine to be used for next tissue sample).
IF NOT Adipose at 95% continue exposure until EITHER tissue identified at 95% confidence level as glandular/fibrous and either benign or malignant OR 60 minutes total exposure time has been reached.

Looking at FIG. 3, this approach is implemented as follows. On initiation of the analysis 32 the desired diagnosis is selected 34, as in the example above, and the conditions and associated confidence levels (e.g. those set out above) are set 36. The irradiation of the sample then commences.

On a regular basis throughout the analysis cycle, the various conditions are checked.

If condition 1 (tissue indicated as being adipose) is met 40, the confidence level of this characterisation is determined 42 and if it meets the desired level A (95%) the analysis stops 44. If not, the analysis continues 38.

If the tissue does not appear to be adipose (i.e. condition 1 is not met), condition 2 is checked 46. In this case, condition 2 is a check to determine whether the maximum 60-minute duration of the analysis has been reached. If it has, the analysis stops 44. Otherwise, it is determined whether confidence level C has been reached 48 (in this example, whether there is 95% confidence that the tissue sample is glandular/fibrous and whether it is benign or malignant). If confidence level C has been attained, the analysis stops 44. Otherwise it continues 38.

Where conditional statements are used, they can be combined with other variables such as patient information (medical history, family history, age etc), for example to determine confidence levels for sub groups—e.g. 95% confidence for older patients in out patients may be acceptable but a 75% (or indeed 99%) confidence level might be required for a young patient undergoing a biopsy or lumpectomy.

These confidence levels may be determined, for instance, through reference to (1) absolute standards or (2) reference databases (e.g. built by analysing a large number of samples).

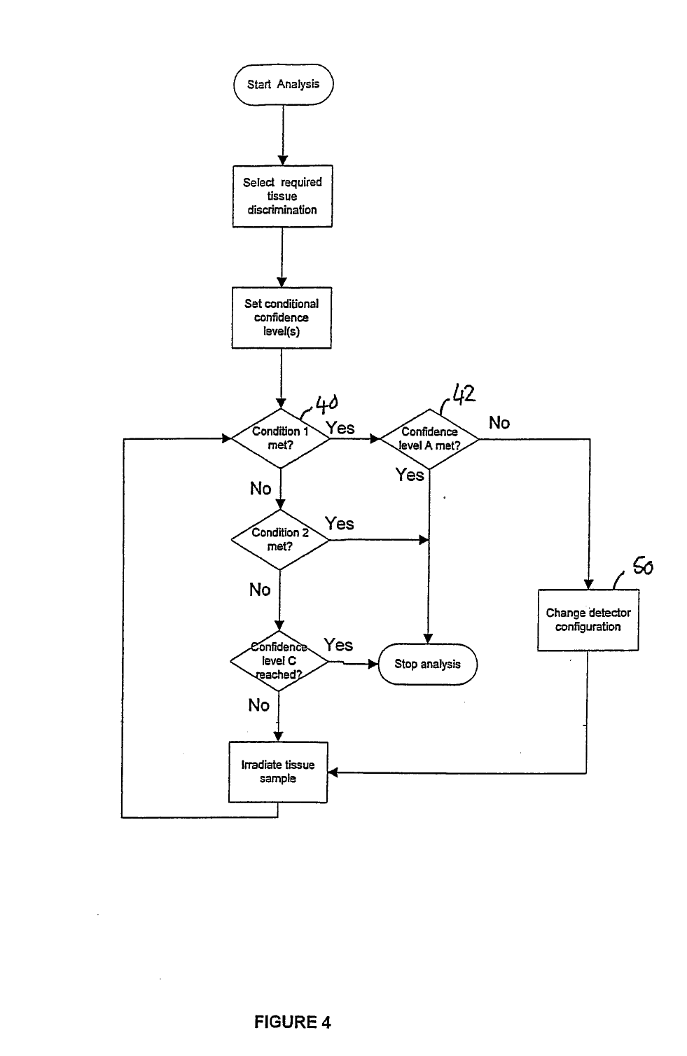

This approach can be extended to cover more complex conditional models, for instance involving further conditions or a more complexly branched decision tree. FIG. 4 illustrates a further exemplary process for controlling payoff between accuracy of diagnosis (confidence level) and duration of the analysis cycle. This example is based closely on the example of FIG. 3 and will not be explained in full. The principle difference here is that in response to condition 1 having been met 40 but confidence level A not having been attained 42, the detector configuration is changed 50.

Put more generally, the conditions set by the operator (directly or indirectly) can be used to determine not only the required confidence levels, but also to control which of a number of possible modes the analysis system operates in.

Taking the system of FIG. 1 as an example, the system may be operable in a number of different modes employing different combinations of one or more of the detectors and/or different configurations (e.g. angles) for each detector. A process in accordance with embodiments of the invention can determine which of these modes to use at which point in the analysis cycle to obtain the optimum payoff between duration and accuracy of the analysis for a given diagnosis or diagnoses.

For example:
(1) Differential diagnosis between adipose and non-adipose may be determined by Compton scattering after 15 minutes; but
(2) Differential diagnosis between benign and malignant at the 75% confidence level may require a combination of Compton, wide and narrow angle for 45 minutes; and
(3) Reaching 95% may require (say) wide and narrow angle to continue to 60 minutes, but Compton may provide no additional information / benefit, so the acquisition of Compton data can be stopped after 45 minutes.

In the various embodiments of the invention, the determination of whether or not a desired confidence level has been met for a given analysis duration can be calculated using empirically derived models (as discussed further above), or algorithms derived from empirical observations, that define a relationship between confidence level and time of analysis, preferably based on a large number of samples having different characteristics. Such models and algorithms can evolve over time as more data is collected.

It will be appreciated that description above is given by way of example and various modifications, omissions or additions to that which has been specifically described can be made without departing from the invention.

1-13. (canceled)

14. A method for analysing a body tissue sample, the method comprising irradiating the tissue sample with penetrating radiation and detecting transmitted or scattered radiation from the sample, wherein the duration of the analysis is determined from one or more desired confidence levels based on a model or algorithm defining a relationship between analysis duration and confidence level.

15. A method according to claim 14, wherein the one or more desired confidence levels are operator selected.

16. A method according to claim 14, wherein the one or more desired confidence levels are automatically set.

17. A method according to claim 14, wherein the one or more desired confidence levels operate as a function of both an operator selection and an automatic setting.

18. A method according to claim 14, wherein the determination of duration is a one-off calculation at the start of the analysis procedure.

19. A method according to claim 14, wherein the determination of duration is a one-off calculation at a predetermined point in the procedure.

20. A method according to claim 14, wherein the determination of duration is adjusted dynamically as the analysis progresses.

21. A method according to claim 14, wherein the determination of duration is adjusted or updated at least twice during the course of the analysis.

22. A method according to claim 14, wherein the model or algorithm is configured to take into account of one or more factors.

23. A method according to claim 22, wherein the one or more factors comprise: histopathology data or histopathological diagnosis; tissue characteristics/types; patient information; or other tissue information.

24. A method for creating or updating a model(s) or an algorithm(s) defining a relationship between analysis duration and confidence level for use in the method of claim 14, wherein the model(s) or algorithm(s) are derived empirically, based on measurements from tissue samples exhibiting a variety of factors.

25. Tissue analysis apparatus for analysing a biological tissue sample, wherein the apparatus comprises a source of penetrating radiation and at least one detector for detecting transmitted or scattered radiation, in use, by a biological sample, and wherein said detector(s) is in communication with a processor that is configured to determine the duration of the analysis of the sample from one or more desired confidence levels based on a model or algorithm defining a relationship between analysis duration and confidence level.

26. Computer software for analysing a biological tissue sample, wherein said software is operative to allow a processor to determine the duration of the analysis of the sample being exposed to a source of radiation based on one or more desired confidence levels based on a model or algorithm defining a relationship between analysis duration and confidence level.

27. A method according to claim 15, wherein the determination of duration is a one-off calculation at the start of the analysis procedure.

28. A method according to claim 16, wherein the determination of duration is a one-off calculation at the start of the analysis procedure.

29. A method according to claim 17, wherein the determination of duration is a one-off calculation at the start of the analysis procedure.
